(19) United States
(12) Patent Application Publication
(10) Pub. No.: US 2022/0193100 A1
(43) Pub. Date: Jun. 23, 2022

(54) COMPOSITION CONTAINING GLUCORAPHANIN AND USE THEREOF

(71) Applicant: SHENZHEN FUSHAN BIOTECH CO., LTD., Guangdong (CN)

(72) Inventors: Xiaolong LI, Guangdong (CN); Zhipeng ZHU, Guangdong (CN); Ligui DENG, Guangdong (CN)

(21) Appl. No.: 17/606,145

(22) PCT Filed: Jun. 11, 2019
(86) PCT No.: PCT/CN2019/090681

§ 371 Date: Oct. 25, 2021

(30) Foreign Application Priority Data
May 8, 2019 (CN) 201910377899.1

(51) Int. Cl. A61K 31/7032; A61K 38/47; A61K 36/31; A61K 31/26

(52) U.S. Cl. CPC A61K 31/7032; A61K 31/26; A61K 36/31; A61K 38/47

(57) ABSTRACT

The present disclosure relates to a composition comprising glucoraphanin and its therapeutic use.

### CROSS REFERENCE TO RELATED APPLICATIONS

This application is a National Stage application of PCT/CN2019/090681, filed on Jun. 11, 2019, which claims priority of Chinese Patent Application No. 201910377899.1, filed on May 8, 2019, and titled with “Composition containing glucoraphanin and use thereof”, and the contents each of which are hereby incorporated by reference in their entirety.

### FIELD

The invention belongs to the field of biomedicine, in particular to a composition comprising glucoraphanin, and the invention also relates to use of the composition in the manufacture of a product for preventing and/or treating diseases or disorders which can be prevented and/or treated by using glucoraphanin.

### BACKGROUND

According to the World Health Organization's 2014 annual report, cancer is the leading cause of morbidity and mortality worldwide, posing a huge threat to people's lives and health, and bringing a heavy economic burden to social development. In the long process of fighting cancer, the concept of cancer chemoprevention was proposed in 1976, which refers to the strategy of using natural or synthetic chemicals to prevent, slow down or reverse the development of cancer.

Sulforaphane (SFN), with chemical name 1-isothiocyanate-4-methanesulfonylbutane, belonging to an isothiocyanate, is a biologically active substance found by Paul Talalay of John Hopkings University from broccoli that has the ability to prevent cancer. It is the strongest anti-cancer ingredient found in vegetables to date. The molecular mechanism of sulforaphane and the results of cell experiments further show that sulforaphane functions as cancer chemoprevention by regulating the phase II enzyme activity for metabolic detoxification of phase I enzyme metabolites or foreign substances (Myzak M C, Dashwood R H. Cancer Lett., 2006, 233:208-18). It is known that sulforaphane is an inducer of Nrf2 (NF-E2 related factor 2, a transcription factor regulating cell oxidative stress). The main mechanism of action is activation of Nrf2 signaling pathway and induction of phase II enzyme (NQO1, glutathione thiotransferase, γ-glutamylcysteine synthetase, glucuronyltransferase, etc.) expression, and regulation of antioxidant response elements and the like. The prior art discloses various activities and effects of sulforaphane and its precursor compound glucoraphanin, for example, as a chemical protective agent against gastric ulcer and Helicobacter pylori infection (CN1935003A; CN1170472C; CN101208079B). Nrf2 is known to be a transcription factor that regulates the expression of many detoxifying enzymes and antioxidant enzymes. It is known that sulforaphane and glucoraphanin have antimicrobial activities against Gram-positive bacteria and Gram-negative bacteria and yeast. Furthermore, they have been shown to exert a protective effect on Parkinson's disease (in a mouse model) and they in particular also have diuretic, anti-anemia and laxative properties. A molecular basis investigation of the mechanism of action of sulforaphane indicates that sulforaphane and glucoraphanin act indirectly as antioxidants by stimulating phase II detoxification enzymes. In addition, sulforaphane and glucoraphanin, which are sulforaphane compounds, have been shown to have UV radiation protection, thereby avoiding sunburn, degradation caused by ROS (reactive oxygen species) and skin cancer (Talalay P; Fahey J W; Healy Z R; Wehage S L; Benedict A L; Min C.; Dinkova-Kostova A T PNAS, 2007, 104, 17500-17505; CN104284885B). Factor Nrf2 has been shown to play an important role in growth factor regulation, signaling and tissue repair (specifically, oxidative stress-induced liver regeneration) in recent years (Beyer T.; Xu W.; Teupser D.; Keller U.; Bugnon P.; Hildt E.; Thiery J.; Yuet Wai K.; Werner S. The EMBO Journal, 2008, 27, 212-223).

In summary, based on the above mechanism, it has been found and confirmed that sulforaphane as an isothiocyanate has various activities and functions associated with its phase II enzyme regulation and Nrf2 activation. With the deepening of research, it has been found that in addition to the role of sulforaphane in the field of cancer chemoprevention, it also has preventive and/or therapeutic effects in many other diseases including diabetes, cardiovascular disease, Helicobacter pylori infection, autism, schizophrenia, depression, and Alzheimer diseases, and it has been validated in animal and clinical trials. For example, sulforaphane can reduce hepatic glucose production in patients with type 2 diabetes and improve glycemic control (Axelsson A S, Tubbs E, Mecham B, et al. Sci Transl Med., 2017, 9 (394)); can reduce vascular inflammation and prevent TNF-α-induced adhesion of monocytes to primary epithelial cells (Nallasamy P, Si H, Babu P V, et al. J Nutr Biochem., 2014, 25(8): 824-33); can inhibit colonization of H. pylori in the stomach of mice and humans, and reduce infection-induced gastric inflammation (Yanaka A, Fahey J W, Fukumoto A, et al. Cancer Prev Res (Phila)., 2009, 2(4): 353-60); can reverse autism-related abnormal symptoms in clinical trials, including oxidative stress, low antioxidant capacity, inhibited glutathione synthesis, decreased mitochondrial function and oxidative phosphorylation, enhanced lipid peroxidation and neuroinflammation (Singh K, Connors S L, Macklin E A, et al. Proc Natl Acad Sci USA, 2014, 111(43): 15550-5); can improve cognitive function in patients with schizophrenia (Shiina A, Kanahara N, Sasaki T, et al. Clin Psychopharmacol Neurosci., 2015, 13(1): 62-7). Broccoli sprouts rich in sulforaphane have a preventive effect on depression (Zhang J C, Yao W, Dong C, et al. J Nutr Biochem., 2017, 39: 134-144). Administering sulforaphane can improve cognitive function of the acute A D mouse model induced by amyloid β-protein (Aβ) in the Y-maze and passive avoidance behavior tests (Kim H V, Kim H Y, Ehrlich H Y, et al. Amyloid, 2013, 20(1): 7-12). In addition, Han Li et al. reported the effect of sulforaphane on pulmonary fibrosis through the Nrf2 pathway (Han Li, Jiang Tao, Chinese Journal of New Drugs and Clinical Medicine, 2016, No. 12).

It is known that cruciferous plants are the main source of sulforaphane and its precursor compound glucoraphanin. Broccoli is a preferred crucifer plant that provides sulforaphane and its precursor compound, glucoraphanin. Broccoli is a Brassicaceae Brassica plant. It is known that the glucoraphanin content is relatively higher in broccoli seeds and seedlings (buds). Even so, it is unrealistic to consume an effective amount of sulforaphane by eating broccoli. Therefore, it is necessary to extract the broccoli and achieve its effective biological efficacy through its extract.

On the other hand, broccoli contains a precursor of sulforaphane, i.e., glucoraphanin, that is not biologically active and requires the myrosinase contained in the plant to decompose glucoraphanin in order to convert it into active sulforaphane. Myrosinase is mainly found in cruciferous plants. Under certain conditions, myrosinase can decompose glucoraphanins to produce products including biologically active isothiocyanates. However, we found that even if glucoraphanin in broccoli is previously converted to sulforaphane by using myrosinase, sulforaphane in the product is unstable to oxygen and heat, so it is difficult be saved and used.

The present inventors have found that if the myrosinase and the glucoraphanin as raw material are separately provided in a solid form and a premix thereof is provided, the decomposition of glucoraphanin by the enzyme during storage can be avoided. After water is added and/or administering is made orally, the decomposition of the enzyme can be achieved in a solution environment, and the effective absorption of sulforaphane can be achieved.

However, during the course of the study, it was unexpected that even for a mixture of myrosinase and glucoraphanin as raw material (including raw materials in the form of broccoli and/or their extracts) present in solid form during storage and placement, there is still a problem of stability, which is manifested in a decrease in the content of glucoraphanin, and also causes problems such as discoloration of the appearance of the product and bitter taste, which affects the quality of the product.

Therefore, there is still a need in the art to overcome the stability problems of compositions comprising glucoraphanin and myrosinase, and to provide a stable composition comprising glucoraphanin and myrosinase with satisfactory quality.

### SUMMARY

The present inventors have unexpectedly found in the study that if a basic salt compound is added to a composition comprising glucoraphanin and myrosinase, the stability of the composition can be well improved while maintaining the appearance and taste stability.

Based on this finding, in a first aspect of the invention, a composition is provided, which comprises the following components:

1) component I providing glucoraphanin;

2) component II providing myrosinase; and

3) a basic salt compound.

In the present invention, the component I providing glucoraphanin may be any substance or raw material capable of providing a source of glucoraphanin compound. Preferably, the component I providing glucoraphanin is selected from the group consisting of cruciferous plants, extracts thereof and mixtures thereof. The cruciferous plant is preferably selected from the group consisting of broccoli, cauliflower, red cabbage, brussels sprouts or cabbage, wherein broccoli is particularly preferred. The crucifer plant may be whole or a part of a plant, such as a whole plant, an aerial part thereof, a flower ball, a seedling, a seed, or a combination thereof. The component I providing glucoraphanin may also be an extract of a cruciferous plant, such as a solvent extract, preferably an aqueous extract, an alcohol extract, a water-alcohol extract. In addition to plant tissues, extracts and mixtures thereof of cruciferous plants, the component I providing glucoraphanin of the present invention may further comprise chemically synthesized, semi-chemically synthesized, and/or enzymatically synthesized glucoraphanin.

In the present invention, the component II providing myrosinase may be any substance or raw material capable of providing a source of myrosinase. Preferably, the component II providing myrosinase is selected from the group consisting of cruciferous plants, extracts thereof, and mixtures thereof. Preferably, the component II providing myrosinase is selected from the group consisting of horseradish, radish and kale. In some preferred embodiments, the component II providing myrosinase is selected from the group consisting of horseradish extract, radish extract, and cabbage extract; in other preferred embodiments, the component II providing myrosinase is a juice or slurry from horseradish, radish, and/or kale, or a powder obtained by drying the juice or slurry.

In the present invention, broccoli means all or a part of a broccoli plant. Preferably, the broccoli is selected from the edible parts of the usual meaning; more preferably, the broccoli is selected from the group consisting of broccoli flower bulb, broccoli seed and broccoli seedling, and combinations thereof.

In the present invention, the broccoli extract means an extract of all or a part of the broccoli plant, including but not limited to an extract of broccoli, the broccoli flower bulb, the broccoli seed and/or an of broccoli seedling. On the other hand, the extract is an extract obtained by extraction with a solvent, and the extract is preferably an aqueous extract, an alcohol extract or a water-alcohol extract, and particularly preferably an aqueous extract.

In the present invention, the component I providing glucoraphanin is preferably selected from the group consisting of broccoli flower bulbs, broccoli seeds, broccoli seedlings, broccoli extracts, and mixtures thereof.

In the present invention, the basic salt compound may be a basic (alkaline) inorganic or organic acid salt, preferably selected from the group consisting of sodium carbonate, potassium carbonate, sodium hydrogencarbonate, potassium hydrogencarbonate, sodium pyrophosphate, sodium citrate, potassium pyrophosphate, potassium citrate and mixtures thereof.

In the present invention, the mass ratio of the component I, the component II and the basic salt compound is 10-80: 1-80:0.1-1; preferably 10-50:1-50:0.1-0.5, more preferably 10-30:1-20:0.1-0.5.

In some preferred embodiments, the composition of the present invention further comprises ascorbic acid.

The composition of the present invention is preferably in a solid form, for example, in the form of a preparation of powder, granule, capsule or tablet. More preferably, in the composition of the present invention, the component I, the component II and the basic salt compound are all present in a solid form. For example, the component I may be in the form of powder (including lyophilized powder) of the extract, seedling or seed.

In some preferred embodiments, the component I of the invention is selected from the group consisting of broccoli seed extract, broccoli seedling powder, broccoli flower bulb lyophilized powder, and mixtures thereof.

In another aspect, the invention provides use of the composition of the invention in the manufacture of a product for prevention and/or treatment of a disease or disorder that can be prevented and/or treated by sulforaphane. The product can be a medicament or food. Preferably, the product is a medicament. In some preferred embodiments, the disease or disorder that can be prevented and/or treated by sulforaphane is selected from the group consisting of cancer, diabetes, cardiovascular disease, Helicobacter pylori infection, autism, schizophrenia, depression, Alzheimer disease (A D) and pulmonary fibrosis.

In another aspect of the invention, a method of converting glucoraphanin to sulforaphane in vitro is provided, which comprises the following steps:

1) providing a composition according to the present invention, and

2) mixing the composition with water or an aqueous solution.

In a third aspect of the invention, a method of supplementing sulforaphane to a subject in need thereof is provided, comprising administering to the subject a composition of the present invention.

In a further aspect, the invention provides a method of preventing and/or treating of a disease or disorder that can be prevented and/or treated by sulforaphane, comprising administering a subject in need thereof a composition of the invention. Preferably, the disease or disorder that can be prevented and/or treated by sulforaphane is selected from the group consisting of cancer, diabetes, cardiovascular disease, Helicobacter pylori infection, autism, schizophrenia, depression, Alzheimer disease (A D) and pulmonary fibrosis.

The present inventors have found that by adding a basic salt compound to the composition, it is possible to avoid a decrease in the content of glucoraphanin in the product and to effectively improve the stability of the composition while maintaining the appearance and taste stability.

### DETAILED DESCRIPTION O F THE INVENTION

### Example 1

(1) Preparation of composition 1 of the present invention: 720 g of broccoli seed aqueous extract (containing 13.0% of glucoraphanin, purchased from Brassica Protection Products LLC, the same as below) was mixed evenly with 296 g of horseradish powder and 10 g of sodium carbonate, to obtain 1.03 kg of Composition 1, wherein glucoraphanin accounted for 9.09%. The above composition 1 was placed in a sachet package in a weight of 5 g per bag to obtain a corresponding powder product.

(2) Preparation of control composition 1: 720 g of broccoli seed water extract (containing 13.0% of glucoraphanin) and 296 g of horseradish powder were mixed evenly to obtain 1.02 kg of the control composition 1, wherein glucoraphanin accounted for 9.18%. The above control composition 1 was placed in a sachet package in a weight of 5 g per bag to obtain a corresponding powder product.

(3) Accelerated stability experiment: The above two powder products were placed in an accelerated test chamber at 37° C. and 75% relative humidity for 3 months, and then taken out, and the appearance change was observed, and the sulforaphane production rate was measured by the following method.

1.03 g of composition 1 and 1.02 g of control composition 1 (both containing glucoraphanin 93.6 mg) were taken, and added to 30 mL of water to incubate the simulated brewing conditions at 37° C. Samples was taken at 5 min, 8 min and 30 min respectively. The sulforaphane content was determined by HPLC and the sulforaphane production rate was calculated.

HPLC method for determination of sulforaphane: The sample solution was taken and passed through a 0.45 μm filter for HPLC analysis. HPLC conditions: column: Huapu Unitary C18 (4.6 mm×250 mm, 5 μm); column temperature: 30° C.; mobile phase: 70% water −30% acetonitrile; flow rate: 0.8 mL/min; injection volume: 10 μL; U V detection wavelength: 245 nm.

The experimental results are shown in Table 1 below.

TABLE 1
Comparison of sulforaphane production rates for Composition
1 and Control Composition 1 powder products

| | sulforaphane | | | | |
|---|---|---|---|---|---|
| | production rate (%) | | | | |
| | 5 | 8 | 30 | | |
| group | min | min | min | appearance | taste |
| Composition 1 | 26 | 38 | 50 | light yellow | own favour |
| (month 0) | | | | powder | |
| Composition 1 | 16 | 25 | 45 | light yellow | own favour |
| (month 3) | | | | powder | |
| Control | 26 | 38 | 49 | light yellow | own favour |
| composition 1 | | | | powder | |
| (month 0) | | | | | |
| Control | 10 | 19 | 31 | pink powder | with |
| composition 1 | | | | | obvious |
| (month 3) | | | | | bitterness |

It can be seen from the experimental results that the composition 1 of the present invention containing the basic salt has no significant change in appearance and taste after the accelerated experiment, and the sulforaphane production rate is kept better than that of the control composition 1 containing no basic salt. The rate of sulforaphane production was basically unchanged before and after the accelerated experiment, which indicated that the sulforaphane content in the composition after hydrolysis was almost unchanged from that before hydrolysis.

### Example 2

(1) Preparation of tablets 1 of the present invention: 200 g of broccoli seedling aqueous extract (containing 13.0% of glucoraphanin), 200 g of horseradish powder, 4 g of vitamin C, 10 g of sodium phosphate and 596 g of tablet excipients (consisting of starch, maltodextrin, and hydroxypropylmethylcellulose, with a ratio (w/w) of 5:80:2, the same below) were evenly mixed, and were tableted based on 0.6 g per tablet, covered with film coating to give 1.02 kg of the tablets 1 of the invention, wherein glucoraphanin accounted for 2.57%. The above-mentioned tablets 1 of the present invention were placed in a bottle with 60 tablets per bottle and a desiccant was added thereto, and the bottle was sealed to obtain a corresponding tablet product.

(2) Preparation of Control Tablet 1: 200 g of aqueous extract of broccoli seedling (containing 13.0% of glucosinolate), 200 g of horseradish powder, 4 g of vitamin C and 596 g of tablet excipients were evenly mixed, and were tableted based on 0.6 g per tablet, covered with film coating to give 1.01 kg of Control Tablet 1, of which glucoraphanin accounted for 2.6%. The above-mentioned tablet 1 of the present invention was placed in a bottle with 60 tablets per bottle and a desiccant was added thereto, and the bottle was sealed to obtain a corresponding tablet product.

(3) Accelerated stability experiment: The above two tablet products were placed in an accelerated test chamber at 37° C. and 75% relative humidity for 3 months, and then taken out, and the appearance change was observed and the sulforaphane production rate was measured by the following method.

30 g of each tablet sample was taken and ground. 1.02 g of the tablet 1 powder of the invention and 1.01 g of the control tablet 1 powder (both containing 26 mg of glucoraphanin) were taken, respectively, and added to a 30 mL artificial postprandial gastric juice simulating solution (the artificial postprandial gastric juice simulating solution was prepared according to Chinese Pharmacopoeia, Part II, 2015 edition, and the pH was adjusted to 3.5), and the temperature was maintained at 37° C. The samples were taken at 30 min and 60 min respectively. The sulforaphane content was determined by HPLC to calculate the sulforaphane production rate. The HPLC method for the determination of sulforaphane was the same as in Example 1.

The experimental results are shown in Table 2 below.

TABLE 2
Comparison of sulforaphane production
rates for Table 1 and Control Table 1

| | sulforaphane | | | |
|---|---|---|---|---|
| | production rate (%) | | | |
| group | 30 min | 60 min | appearance | taste |
| Tablet 1 of the | 17.5 | 23.0 | light white | own favour |
| present invention | | | tablet | |
| (month 0) | | | | |
| Tablet 1 of the | 11.1 | 16.4 | light white | own favour |
| present invention | | | tablet | |
| (month 3) | | | | |
| Control tablet 1 | 17.6 | 23.3 | light white | own favour |
| (month 0) | | | tablet | |
| Control tablet 1 | 7.9 | 11.9 | pink tablet | with |
| (month 3) | | | | obvious |
| | | | | bitterness |

It can be seen from the experimental results that the tablet 1 of the present invention containing the basic salt has no significant change in appearance and taste after the accelerated experiment, and the rate of sulforaphane production remains well compared to the control tablet 1 containing no basic salt. After the accelerated stability experiment, the rate of sulforaphane production was significantly higher than that of the control tablets.

### Example 3

(1) Preparation of tablets 2 of the present invention: 200 g of broccoli seed aqueous extract (containing 13.0% of glucoraphanin), 50 g broccoli seedling powder (containing 4.5% of glucoraphanin), 50 g broccoli flower bulb lyophilized powder, 250 g horseradish powder, 4 g of calcium vitamin C, 10 g of sodium citrate and 596 g of tablet excipients (consisting of starch, maltodextrin, and hydroxypropylmethylcellulose, with a ratio (w/w) of 5:80:2) were evenly mixed, and were tableted based on 0.6 g per tablet, covered with film coating to give 1.16 kg of the tablets 2 of the invention, wherein glucoraphanin accounted for 2.80%. The above-mentioned tablets 2 of the present invention were placed in a bottle with 60 tablets per bottle and a desiccant was added thereto, and the bottle was sealed to obtain a corresponding tablet product.

(2) Preparation of Control Tablet 2: 200 g of aqueous extract of broccoli seeds (containing 13.0% of glucosinolate), 50 g broccoli seedling powder (containing 4.5% of glucoraphanin), 50 g broccoli flower bulb lyophilized powder, 250 g horseradish powder, 4 g of calcium vitamin C, 10 g of sodium phosphate and 596 g of tablet excipients (consisting of starch, maltodextrin, and hydroxypropylmethylcellulose, with a ratio (w/w) of 5:80:2) were evenly mixed, and were tableted based on 0.6 g per tablet, covered with film coating to give 1.15 kg of the control tablets 2 of the invention, wherein glucoraphanin accounted for 2.83%. The above-mentioned control tablets 2 of the present invention were placed in a bottle with 60 tablets per bottle and a desiccant was added thereto, and the bottle was sealed to obtain a corresponding control tablet product.

(3) Accelerated stability experiment: The above two tablet products were placed in an accelerated test chamber at 37° C. and 75% relative humidity for 3 months, and then taken out, and the appearance change was observed and the sulforaphane production rate was measured by the following method.

100 g of each tablet sample was taken and ground. 1.16 g of the tablet 2 powder of the invention and 1.15 g of the control tablet 2 powder (both containing 28 mg of glucoraphanin) were taken, respectively, and added to a 30 mL artificial postprandial gastric juice simulating solution (the artificial postprandial gastric juice simulating solution was prepared according to Chinese Pharmacopoeia, Part II, 2015 edition, and the pH was adjusted to 3.5), and the temperature was maintained at 37° C. The samples were taken at 30 min and 60 min respectively. The sulforaphane content was determined by HPLC to calculate the sulforaphane production rate. The HPLC method for the determination of sulforaphane was the same as in Example 1.

The experimental results are shown in Table 3 below.

TABLE 3
Comparison of sulforaphane production
rates for Table 2 and Control Table 2

| | sulforaphane | | | |
|---|---|---|---|---|
| | production rate (%) | | | |
| group | 30 min | 60 min | appearance | taste |
| Tablet 2 of the | 24.7 | 38.2 | light white | own favour |
| present invention | | | tablet | |
| (month 0) | | | | |
| Tablet 2 of the | 14.1 | 22.6 | light white | own favour |
| present invention | | | tablet | |
| (month 3) | | | | |
| Control tablet 2 | 24.7 | 38.2 | light white | own favour |
| (month 0) | | | tablet | |
| Control tablet 2 | 9.2 | 15.3 | pink tablet | with |
| (month 3) | | | | obvious |
| | | | | bitterness |

It can be seen from the experimental results that the tablet 2 of the present invention containing the basic salt has no significant change in appearance and taste after the accelerated experiment, and the rate of sulforaphane production remains well compared to the control tablet 2 containing no basic salt. After the accelerated stability experiment, the rate of sulforaphane production was significantly higher than that of the control tablets.

1. A composition, comprising the following components:
1) component I providing glucoraphanin;
2) component II providing myrosinase; and
3) a basic salt compound.

2. The composition according to claim 1, further comprising ascorbic acid.

3. The composition according to claim 1, wherein the component I is selected from the group consisting of cruciferous plants, extracts thereof, and mixtures thereof.

4. The composition according to claim 1, wherein the cruciferous plant is broccoli.

5. The composition according to claim 3, wherein the cruciferous plant is whole of the plant, a part of the plant, or a mixture thereof.

6. The composition according to claim 3, wherein the cruciferous plant is selected from the group consisting of flower bulbs, seeds, sprouts and mixtures thereof.

7. The composition according to claim 1, wherein the basic salt compound is selected from the group consisting of sodium carbonate, potassium carbonate, sodium hydrogencarbonate, potassium hydrogencarbonate, sodium pyrophosphate, sodium citrate, potassium pyrophosphate, potassium citrate and mixtures thereof.

8. The composition according to claim 1, wherein the mass ratio of the component I, the component II and the basic salt compound is 10-80:1-80:0.1-1.

9. The composition according to claim 1 which is in form of solid.

10. The composition according to claim 1, which is in form of a preparation of powder, granule, capsule or tablet.

11. The composition according to claim 1, wherein the component I is selected from the group consisting of broccoli seed extract, broccoli sprout powder, broccoli flower bulb lyophilized powder, and mixtures thereof.

12. The composition according to claim 1, wherein the component II is selected from the group consisting of horseradish, radish, kale, a juice or a slurry thereof, and an extract thereof.

13. A method of preventing and/or treating a disease or disorder that can be prevented and/or treated by glucoraphanin, comprising administering the composition according to claim 1 to a subject in need thereof.

14. The method according to claim 13, wherein the disease or disorder that can be prevented and/or treated by glucoraphanin is selected from the group consisting of cancer, diabetes, cardiovascular diseases, Helicobacter pylori infection, autism, schizophrenia, depression, Alzheimer's disease and pulmonary fibrosis.

15. A method of converting glucoraphanin to sulforaphane in vitro, comprises the steps of:
1) providing a composition according to claim 1, and
2) mixing the composition with water or an aqueous solution.

16. A method of supplementing sulforaphane to a subject in need thereof, comprising administering to the subject a composition according to claim 1.
